(19) United States
(12) Patent Application Publication
(10) Pub. No.: US 2025/0295372 A1
(43) Pub. Date: Sep. 25, 2025

(54) MICROVASCULAR FLOW ULTRASONIC IMAGING METHOD AND SYSTEM

(71) Applicant: FUDAN UNIVERSITY, Shanghai (CN)

(72) Inventors: Kailiang XU, Shanghai (CN); Shuangyi CHENG, Shanghai (CN); Junjin YU, Shanghai (CN); Yapeng FU, Shanghai (CN); Xingyi GUO, Shanghai (CN)

(21) Appl. No.: 18/861,780

(22) PCT Filed: Aug. 31, 2023
(86) PCT No.: PCT/CN2023/116211

§ 371 Date: Oct. 30, 2024

(30) Foreign Application Priority Data
Sep. 5, 2022 (CN) 202211080231.9

(51) Int. Cl. A61B 8/06; A61B 8/00; A61B 8/14

(52) U.S. Cl. CPC A61B 8/06; A61B 8/145; A61B 8/5215

(57) ABSTRACT

A microvascular flow ultrasonic imaging method and system are provided. The method comprises: includes constructing a combined sequence having a linear imaging sequence and a nonlinear imaging sequence; transmitting the combined sequence to an imaging area and acquiring multiple groups of echo signals within a preset time period to form an echo signal group sequence; sequentially carrying out nonlinear filtering processing and beamforming on each group of echo signals in the echo signal group sequence to obtain a corresponding nonlinear ultrasound image sequence; identifying microbubbles in each frame of image of the nonlinear ultrasound image sequence frame by frame, and tracking a trajectory of the microbubbles according to identifying and positioning results, a microbubble trajectory being determined by identifying and positioning results of microbubbles in continuous N frames of images in the image sequence; and reconstructing and obtaining a super-resolution microvascular flow image based on the tracked microbubble trajectory.

### FIELD

This invention relates to the field of super resolution ultrasound imaging, particularly to super resolution nonlinear ultrasound microvascular imaging technology.

### BACKGROUND

In recent years, inspired by optical localization microscopy, the medical ultrasound community has developed a new technology called Ultrasound Localization Microscopy (ULM). This technology combines the advantages of high resolution and deep penetration, enabling sub-wavelength resolution microvascular angiography of deep human tissues (such as brain, spinal cord, heart, kidney, etc.). It has significantly promoted the development of in vivo deep microvascular angiography. Compared to conventional ultrasound and ultrafast ultrasound imaging, ULM can achieve sub-wavelength (approaching one-tenth of a wavelength, with vessel diameter <10 μm) microvascular imaging. Efficient detection and precise localization of microbubble signals are prerequisites for accurate reconstruction of super-resolution ULM images. Currently, spatio-temporal linear filtering methods represented by Singular value decomposition (SVD) have been widely used for extracting microbubble signal components. Through SVD decomposition of spatio-temporal signal variations, linear filtering methods can effectively extract the “high-velocity moving” microbubble echo signal components.

However, microbubbles in fine blood vessels often exhibit “low-velocity movement” characteristics, with relatively small amplitude of related spatio-temporal variation component signals. As a result, linear ultrasound imaging and related filtering techniques are still not suitable for detecting “low flow velocity” microbubble signals. This bottleneck problem limits the further improvement of ULM technology in resolution and imaging accuracy, as well as its invention in “low flow velocity” microvascular angiography.

### SUMMARY

The purpose of this invention is to provide a microvascular blood flow ultrasound imaging method and system, which breaks through the limitations of ULM in low flow velocity microvascular imaging, can obtain microvascular blood flow information across all velocity ranges, and improves its imaging accuracy.

This invention discloses a microvascular blood flow ultrasound imaging method, including the following steps:
(a) constructing a contrast pulse sequence containing linear imaging sequences and nonlinear imaging sequences;
(b) transmitting the contrast pulse sequence to an imaging area, and acquiring multiple sets of echo signals within a preset time period to form an echo signal group sequence, wherein blood vessels in the imaging area are injected with ultrasound microbubbles;
(c) performing nonlinear filtering processing and beamforming on the echo signals in the echo signal group sequence to obtain a corresponding nonlinear ultrasound image sequence;
(d) identifying and locating microbubbles frame by frame in each frame of the nonlinear ultrasound image sequence, and tracking microbubble trajectories based on identification and localization results, wherein a microbubble trajectory is determined through the identification and localization results of microbubbles in N consecutive frames of the image sequence;
(e) reconstructing a super-resolution microvascular blood flow image based on the tracked microbubble trajectories.

In a preferred example, the step (c) further comprises: sequentially performing linear filtering processing and beamforming on each set of echo signals in the echo signal group sequence, and sequentially performing nonlinear filtering processing and beamforming on each set of echo signals in the echo signal group sequence, to obtain a corresponding linear ultrasound image sequence and nonlinear ultrasound image sequence;
the step (d) further comprises: identifying and locating microbubbles in each of the linear ultrasound image sequence and the nonlinear ultrasound image sequence on a frame-by-frame basis, tracking microbubble trajectories based on identification and location results, and determining and integrating duplicate microbubble trajectories in the time-aligned linear ultrasound images of the linear ultrasound image sequence and nonlinear ultrasound images of the nonlinear ultrasound image sequence into a new trajectory;
the step (e) further comprises: reconstructing a super-resolution microvascular blood flow image based on the tracked microbubble trajectories and the integrated new trajectories.

In a preferred example, the determining and integrating duplicate microbubble trajectories in the time-aligned linear ultrasound images of the linear ultrasound image sequence and nonlinear ultrasound images of the nonlinear ultrasound image sequence into a new trajectory, further comprises:
calculating a corresponding velocity for each microbubble trajectory based on tracking results;
if the absolute value of the velocity difference between two trajectories in the time-aligned linear ultrasound images of the linear ultrasound image sequence and nonlinear ultrasound images of the nonlinear ultrasound image sequence is less than a first preset threshold, and the average Euclidean distance of the point-by-point positions of the two trajectories is less than a second preset threshold, determining the two trajectories as the duplicate microbubble trajectories and integrating them into a new trajectory.

In a preferred example, when sequentially performing nonlinear filtering processing on each set of echo signals in the echo signal group sequence, the method further comprises:

Performing Fourier transform on each set of echo signals respectively: P₁[ω]=Σₙ₌₀^{N}p₁e^{-jωn}, P₂[ω]=Σₙ₌₀^{N}p₂[n]e^{-jωn}, where p₁[n] and p₂[n] are echo signals after two transmissions of ultrasound waves respectively, ω is a discrete frequency, n is a discrete time, and N is a number of sampling points for each reception;
extracting the fundamental frequency and its nearby components P′₁[ω] and P′₂[ω] of the Fourier transformed echo signals: P′₁[ω]=P′₁[ω]|_{ωϵ[ω}_{₀}_{-Δω,ω}_{₀}_{+Δω]}, P′₂[ω]=P′₂ [ω]|_{ωϵ[ω}_{₀}_{-Δω,ω}_{₀}_{+Δω]}, where ω₀ is the fundamental frequency during transmission and reception, and Δω is the half bandwidth;
using the least squares method or gradient descent method to calculate the fundamental wave amplitude correction coefficient θ_{ω}_{₀} between the echo signals of the set that minimizes the difference Σ_{ω}(P′₁[ω]−θ_{ω}_{₀}P′₂[ω])² between P′₁[ω]and P′₂[ω], and applying the fundamental wave amplitude correction coefficient θ_{ω}_{₀} to p₂[n] to obtain p′₂[n]=θ₁₀₇ _{₀}p₂[n], denoted as the echo signal after fundamental wave amplitude correction of p₂[n], where ωₛ is the maximum sampling frequency.

In a preferred example, the nonlinear imaging sequence comprises linear sequence and modulation sequence pairs, the modulation sequence in the linear sequence and modulation sequence pair is obtained by performing a preset modulation method on the linear sequence, wherein the preset modulation method comprises one or more of the following: pulse inversion, amplitude modulation, amplitude-phase modulation.

In a preferred example, the nonlinear imaging sequence comprises multiple identical pulse signals;

when transmitting the contrast pulse sequence to the imaging area, further comprising: dividing ultrasound array elements into multiple groups, and transmitting the multiple identical pulse signals to the imaging area by alternating transmission of the multiple groups.

In a preferred example, the sampling frequency for transmitting the contrast pulse sequence and receiving echoes comprises the Nyquist frequency.

This invention also discloses a microvascular blood flow ultrasound imaging system comprising:
a contrast pulse sequence construction module, for constructing a contrast pulse sequence containing linear imaging sequences and nonlinear imaging sequences;
a transmission and reception module, for transmitting the contrast pulse sequence to an imaging area, and acquiring multiple sets of echo signals within a preset time period to form an echo signal group sequence, wherein the blood vessels in the imaging area are injected with ultrasound microbubbles;
a nonlinear filtering and beamforming module, for sequentially performing nonlinear filtering processing and beamforming on each set of echo signals in the echo signal group sequence to obtain a corresponding nonlinear ultrasound image sequence;
a microbubble trajectory tracking module, for identifying and locating microbubbles frame by frame in each frame of the nonlinear ultrasound image sequence, and tracking microbubble trajectories based on identification and localization results, wherein a microbubble trajectory is determined through the identification and localization results of microbubbles in N consecutive frames of the image sequence;
a microvascular blood flow image reconstruction module, for reconstructing a super-resolution microvascular blood flow image based on the tracked microbubble trajectories.

In a preferred embodiment, the system further comprises a linear filtering and beamforming module, the linear filtering and beamforming module is used for sequentially performing linear filtering processing and beamforming on each set of echo signals in the echo signal group sequence to obtain a corresponding linear ultrasound image sequence;
the microbubble trajectory tracking module is further used for identifying and locating microbubbles frame by frame in each frame of the linear ultrasound image sequence and the nonlinear ultrasound image sequence respectively, and tracking microbubble trajectories based on the identification and localization results;
the system further comprises a microbubble trajectory integration module, which is used for determining duplicate microbubble trajectories in time-aligned linear ultrasound images and nonlinear ultrasound images of the two image sequences and integrating them into a new trajectory;
the microvascular blood flow image reconstruction module is further used for reconstructing a super-resolution microvascular blood flow image based on the tracked microbubble trajectories and integrated new trajectories.

In a preferred embodiment, the microbubble trajectory integration module is further configured to calculate corresponding velocities based on the tracking results of each microbubble trajectory respectively, and if the absolute value of the velocity difference between two trajectories in time-aligned linear ultrasound images and nonlinear ultrasound images of the two image sequences is less than a first predetermined threshold, and the average value of the Euclidean distances of the point-by-point positions of these two trajectories is less than a second predetermined threshold, then determine these two trajectories as the duplicated microbubble trajectories and integrate them into a new trajectory.

The present invention also discloses a microvascular blood flow ultrasound imaging method comprising:
(a) constructing a forming sequence containing linear imaging sequences and nonlinear imaging sequences;
(b) transmitting the forming sequence to an imaging area and imaging based on echoes, and acquiring an ultrasound image sequence within a preset time period, wherein the blood vessels in the imaging area are injected with ultrasound microbubbles;
(c) sequentially performing nonlinear filtering processing on each frame of the ultrasound image sequence to obtain a corresponding nonlinear ultrasound image sequence;
(d) identifying and locating microbubbles frame by frame in each frame of the nonlinear ultrasound image sequence, and tracking microbubble trajectories based on identification and localization results, wherein a microbubble trajectory is determined through the identification and localization results of microbubbles in N consecutive frames of the image sequence;
(e) reconstructing a super-resolution microvascular blood flow image based on the tracked microbubble trajectories.

In a preferred embodiment, the step (c) further comprises: sequentially performing linear filtering processing on each frame of the ultrasound image sequence to obtain a corresponding linear ultrasound image sequence, and sequentially performing nonlinear filtering processing on each frame of the ultrasound image sequence to obtain a nonlinear ultrasound image sequence;
the step (d) further comprises: identifying and locating microbubbles in each of the linear ultrasound image sequence and the nonlinear ultrasound image sequence on a frame-by-frame basis, and tracking microbubble trajectories based on identification and location results; determining and integrating duplicate microbubble trajectories in the time-aligned linear ultrasound images of the linear ultrasound image sequence and nonlinear ultrasound images of the nonlinear ultrasound image sequence into a new trajectory;
the step (e) further comprises: reconstructing a super-resolution microvascular blood flow image based on the tracked microbubble trajectories and integrated new trajectories.

In a preferred embodiment, he determining and integrating duplicate microbubble trajectories in the time-aligned linear ultrasound images of the linear ultrasound image sequence and nonlinear ultrasound images of the nonlinear ultrasound image sequence into a new trajectory, wherein a microbubble trajectory is determined through the identification and localization results of microbubbles in N consecutive frames of the image sequence, further comprises:
calculating a corresponding velocity for each microbubble trajectory based on the tracking results;
if the absolute value of the velocity difference between two trajectories in time-aligned linear ultrasound images and nonlinear ultrasound images of the two image sequences is less than a first predetermined threshold, and the average value of the Euclidean distances of the point-by-point positions of these two trajectories is less than a second predetermined threshold, then determining these two trajectories as the duplicated microbubble trajectories and integrating them into a new trajectory.

In a preferred embodiment, when sequentially performing nonlinear filtering processing on each group of echo signals in the echo signal group sequence, it further comprises:
performing Fourier transform in the axial direction on each frame of image obtained by beamforming: M₁[ω]=Σₙ₌₀^{N}m₁[n]e^{-jωn}, M₂[ω]=Σₙ₌₀^{N}m₂[n]e^{-jωn}, where m₁[n] and m₂[n] are the column signals of the images after two transmissions of ultrasound waves respectively, ωis the discrete frequency, n is the discrete time, and N is the number of sampling points for each reception;
extracting the fundamental frequency and its nearby components M′₁[ω] and M′₂[ω] of the Fourier transformed column (axial) of the image: M′₁[ω]=M₁[ω]|_{ωϵ[ω}_{₀}_{-Δω,ω}_{₀}_{+Δω]}, M′₂[ω]=M₂[ω]|_{ωϵ[ω}_{₀}_{-Δω,ω}_{₀}_{+Δω]},where ω₀ is the fundamental frequency during transmission and reception, and Δω is the half bandwidth;
using the least squares method or gradient descent method to calculate the fundamental wave amplitude correction coefficient θ_{ω}_{₀} between the column (axial) of the set of images that minimizes the difference Σ_{ω}(M′₁[ω]−θ_{ω}_{₀}M′₂[ω])² between M′₁[ω] and M′₂[ω], and applying the fundamental wave amplitude correction coefficient θ_{ω}_{₀} to m₂[n] to obtain m′₂[n]=θ_{ω}_{₀}m₂[n], denoted as the column signal of the image after fundamental wave amplitude correction of m₂[n], where ωₛ is the maximum sampling frequency.

In a preferred embodiment, the nonlinear imaging sequence comprises linear sequence and modulation sequence pairs, the modulation sequence in the linear sequence and modulation sequence pair is obtained by performing a preset modulation method on the linear sequence, wherein the preset modulation method comprises one or more of the following: pulse inversion, amplitude modulation, amplitude-phase modulation.

In a preferred embodiment, the nonlinear imaging sequence comprises multiple identical pulse signals; when transmitting the contrast pulse sequence to the imaging area, further comprising: dividing ultrasound array elements into multiple groups, and transmitting the multiple identical pulse signals to the imaging area by alternating transmission of the multiple groups.

In a preferred embodiment, the sampling frequency for transmitting the contrast pulse sequence and receiving echoes comprises the Nyquist frequency.

The present invention also discloses a microvascular blood flow ultrasound imaging system comprising:
a contrast pulse sequence construction module, configured to construct a contrast pulse sequence containing a linear imaging sequence and a nonlinear imaging sequence;
an ultrasound imaging module, configured to transmit the contrast pulse sequence to an imaging area and perform imaging based on echoes, and obtain an ultrasound image sequence within a preset time period, wherein ultrasound microbubbles are injected into blood vessels in the imaging area;
a nonlinear filtering module, configured to perform nonlinear filtering processing on each frame of image in the ultrasound image sequence to obtain a corresponding nonlinear ultrasound image sequence;
a microbubble trajectory tracking module, configured to identify and locate microbubbles in each frame of image of the nonlinear ultrasound image sequence frame by frame, and track microbubble trajectories based on the identification and localization results, wherein one microbubble trajectory is determined by the identification and localization results of microbubbles in N consecutive frames of images in the image sequence;
a microvascular blood flow image reconstruction module, configured to reconstruct a super-resolution microvascular blood flow image based on the tracked microbubble trajectories.

In a preferred example, the system further comprises a linear filtering module, configured to perform linear filtering processing on each frame of image in the ultrasound image sequence to obtain a corresponding linear ultrasound image sequence;
the microbubble trajectory tracking module is further configured to identify and locate microbubbles in each frame of image frame by frame for both the linear ultrasound image sequence and the nonlinear ultrasound image sequence, and track microbubble trajectories based on the identification and localization results;
the system further includes a microbubble trajectory integration module, configured to determine duplicated microbubble trajectories in time-aligned linear ultrasound images and nonlinear ultrasound images of the two image sequences and integrate them into a new trajectory;
the microvascular blood flow image reconstruction module is further configured to reconstruct a super-resolution microvascular blood flow image based on the tracked microbubble trajectories and the integrated new trajectories.

In a preferred example, the microbubble trajectory integration module is further configured to calculate a corresponding velocity for each microbubble trajectory based on its tracking result, and if the absolute value of the velocity difference between two trajectories in time-aligned linear ultrasound images and nonlinear ultrasound images of the two image sequences is less than a first predetermined threshold, and the average Euclidean distance of the point-by-point positions of these two trajectories is less than a second predetermined threshold, then these two trajectories are determined as the duplicated microbubble trajectories and integrated into a new trajectory.

The present invention also discloses a microvascular blood flow ultrasound imaging device comprising:
a memory, configured to store computer executable instructions; and
a processor, configured to implement the steps in the method described above when executing the computer executable instructions.

The present invention also discloses a computer-readable storage medium storing computer executable instructions, wherein the computer executable instructions, when executed by a processor, implement the steps in the method described above.

The embodiments of the present invention include at least the following advantages:
(1) By exciting the nonlinearity of microbubbles through contrast pulse sequences for nonlinear ultrasound imaging, the echo signal detection capability of slowly moving microbubbles is enhanced, compensating for the deficiency of common ultrasound localization microscopy in detecting low-velocity microbubbles. Given that slowly moving microbubbles often appear in finer blood vessels, this improvement can enhance the spatial resolution of angiography.
(2) By using contrast pulse sequences of linear imaging sequences and nonlinear imaging sequences to perform imaging scans on the imaging area, and performing linear and nonlinear filtering processing on echo signals or imaging images respectively, tracking microbubble trajectories, deduplicating, and reconstructing microvascular blood flow images based on the deduplicated microbubble trajectories, it is ultimately possible to reconstruct microvascular blood flow information and velocity vector maps for all velocity ranges. Furthermore, by combining linear imaging sequences and nonlinear imaging sequences, multiple results can be obtained for each frame of image, which is equivalent to increasing the frame rate of ultrasound imaging.
(3) Considering that the linear components of tissue signals are mainly concentrated at the fundamental frequency and its vicinity, a set of linear deviation coefficients between echoes can be found through matching of the fundamental frequency and its nearby components, and by correcting the linear component deviation between echoes of any set of ultrasound sequences, only the nonlinear microbubble echo components are retained after filtering.

This specification contains numerous technical features distributed among various technical solutions. Listing all possible combinations of these technical features (i.e., technical solutions) would make the specification too lengthy. To avoid this problem, the technical features disclosed in the above-mentioned invention content, the technical features disclosed in various embodiments and examples in the following text, and the technical features disclosed in the drawings can all be freely combined to form various new technical solutions (which should be considered as already disclosed in this specification), unless such combinations of technical features are technically infeasible. For example, if feature A+B+C is disclosed in one example and feature A+B+D+E is disclosed in another example, and features C and D are equivalent technical means that serve the same purpose, only one of them can be chosen for technical reasons and cannot be used simultaneously. Feature E can be combined with feature C from a technical perspective. Therefore, the A+B+C+D solution should not be considered as already disclosed because it is technically infeasible, while the A+B+C+E solution should be considered as already disclosed.

### BRIEF DESCRIPTION OF THE DRAWINGS

FIG. 1 is a flow diagram of a microvascular blood flow ultrasound imaging method according to the first embodiment of the present invention.

FIG. 2 is a structural diagram of a microvascular blood flow ultrasound imaging system according to the second embodiment of the present invention.

FIG. 3 is a flow diagram of a microvascular blood flow ultrasound imaging method according to the third embodiment of the present invention.

FIG. 4 is a structural diagram of a microvascular blood flow ultrasound imaging system according to the fourth embodiment of the present invention.

FIG. 5 is a schematic diagram of a contrast pulse sequence including linear imaging sequences and nonlinear imaging sequences according to an example of the present invention.

FIG. 6 is a spectral analysis diagram of useful microbubble signals obtained by the nonlinear filtering module according to an example of the present invention.

FIG. 7 is a B-mode image of microbubbles obtained by nonlinear ultrasound imaging according to an example of the present invention.

FIG. 8 is a linear B-mode image of microbubbles under background noise according to an example of the present invention.

FIG. 9 is a nonlinear pulse inversion B-mode image of microbubbles under background noise according to an example of the present invention.

FIG. 10 is a nonlinear amplitude modulation B-mode image of microbubbles under background noise according to an example of the present invention.

FIG. 11 is a nonlinear amplitude-phase modulation B-mode image of microbubbles under background noise according to an example of the present invention.

FIG. 12 is a nonlinear odd-even alternating B-mode image of microbubbles under background noise according to an example of the present invention.

FIGS. 13A and 13B are linear B-mode images and nonlinear odd-even alternating B-mode images of five microbubbles under background noise, respectively, according to an example of the present invention.

FIG. 14 is a super-resolution ultrasound microvascular blood flow vector result image of rat spinal cord obtained based on microbubble trajectories under linear filtering in the proposed microvascular blood flow ultrasound imaging method in example 3 of the present invention.

FIG. 15 is a super-resolution color ultrasound microvascular blood flow vector result image of rat spinal cord obtained based on microbubble trajectories under nonlinear filtering in the proposed microvascular blood flow ultrasound imaging method in example 3 of the present invention.

FIG. 16 shows the super-resolution ultrasound microvascular blood flow vector result image of rat spinal cord obtained by integrating microbubble trajectories after linear filtering and nonlinear filtering in the microvascular blood flow ultrasound imaging method proposed in example 3 of the present invention.

### DETAILED DESCRIPTION

In the following description, numerous technical details are set forth in order to provide a thorough understanding of the present invention. However, those skilled in the art can understand that the technical solution claimed in this invention can be realized without these technical details and various changes and modifications based on the following embodiments.

Explanation of some concepts:

Sequence: Multiple transmissions and receptions are usually required to generate one frame of ultrasound image. For example, the linear imaging sequence in this invention requires at least one transmission and reception process, while the nonlinear imaging sequence requires multiple transmissions and receptions combined according to the contrast multi-pulse imaging strategy. Similarly, combining linear and nonlinear sequences can achieve better imaging effects, and such a sequence is called a contrast pulse sequence.

Nonlinear ultrasound imaging: Ultrasound waves propagating in tissues or microbubbles cause nonlinear oscillations and thus generate harmonics. Nonlinear ultrasound imaging refers to imaging using nonlinear harmonic components in ultrasound echo signals, also known as harmonic imaging, which is an ultrasound technique that can improve image clarity. Harmonic imaging is mainly divided into tissue harmonic imaging and contrast-enhanced (microbubble) harmonic imaging. Tissue harmonic imaging uses harmonic components in echoes for imaging by utilizing the nonlinear distortion that occurs when ultrasound waves propagate in tissues. With the continuous development of microbubble material technology, microbubbles exhibit much stronger nonlinear characteristics than tissues, leading to the development of contrast-enhanced harmonic imaging using nonlinear echoes from microbubbles. At low mechanical index, microbubbles are not destroyed, so harmonic imaging provides a new method for real-time imaging of microbubble contrast agents in microcirculation and large blood vessels: at low power, the response of microbubbles is nonlinear, while sound propagation in tissues is basically linear. The key to distinguishing tissues and microbubbles is to preferentially detect nonlinear echoes from microbubbles while eliminating background tissue signals.

Super-resolution ultrasound imaging: Microbubbles as ultrasound contrast agents are introduced into blood vessels through intravenous injection. Based on B-mode images obtained by ultrafast ultrasound imaging units, corresponding super-resolution vascular blood flow images are obtained through clutter filtering, ultrasound microbubble localization tracking, and super-resolution reconstruction; or without intravenous injection of ultrasound contrast agents (microbubbles), corresponding super-resolution microvascular blood flow ultrasound images are obtained through clutter filtering, microbubble localization tracking, and super-resolution reconstruction based on B-mode images obtained by ultrafast ultrasound imaging units.

Clutter filtering: The received echo data contains echo signals from static tissues, blood flow echo signals, and noise. To clearly observe microvasculature in images, noise and static tissue signal data need to be filtered out from image data. Currently commonly used methods include high-pass filtering, adaptive filtering, singular value decomposition, robust principal component analysis, independent component analysis, etc.

Blood flow vector imaging: Arrows with directions are used to represent blood flow direction and velocity. Common methods include Vector Doppler, which deduces the true blood flow velocity and direction through axial blood flow velocities under different angle plane waves. Other methods include Speckle Tracking, etc.

In order to make the objectives, technical solutions, and advantages of the present invention clearer, embodiments of the present invention will be further described in detail below with reference to the drawings.

### First Embodiment

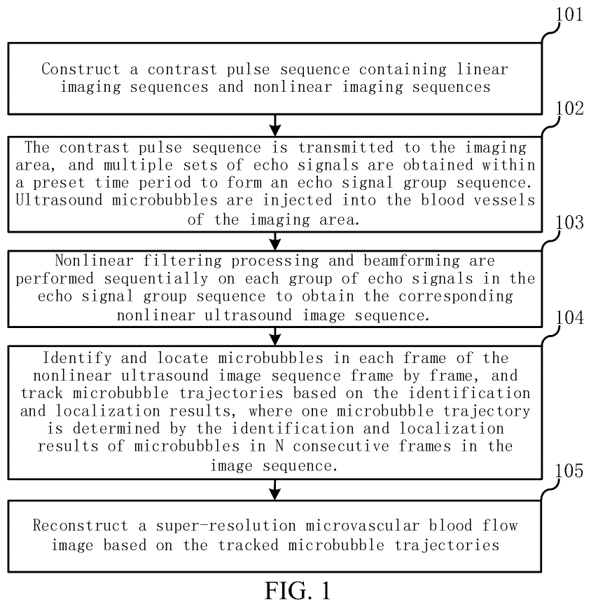

The first embodiment of this invention relates to a microvascular blood flow ultrasound imaging method, the process of which is shown in FIG. 1. The method includes the following steps:

In step 101, a contrast pulse sequence containing linear imaging sequences and nonlinear imaging sequences is constructed. Then, proceed to step 102, where the contrast pulse sequence is transmitted to the imaging area, and multiple sets of echo signals are obtained within a preset time period to form an echo signal group sequence. Ultrasound microbubbles are injected into the blood vessels of the imaging area.

Optionally, in step 102, plane wave imaging, focused wave imaging, diverging wave imaging, synthetic aperture imaging, etc. can be performed on the imaging area using the contrast pulse sequence, but not limited to these.

In one embodiment, nonlinear contrast pulses are obtained by modulating linear sequences, and nonlinear imaging is performed by transmitting contrast pulse sequences containing these contrast pulses to the imaging area. Optionally, the nonlinear imaging sequence includes linear sequence and modulation sequence pairs, where the modulation sequence in the linear sequence and modulation sequence pair is obtained by performing a preset modulation method on the linear sequence. The preset modulation method includes one or more of the following: pulse inversion, amplitude modulation, amplitude-phase modulation. Among them, pulse inversion: includes two waves s1(t) and s2(t), s2(t)=−s1(t); amplitude modulation: includes two waves s1(t) and s2(t), s2(t)=a·s1(t); amplitude-phase modulation: includes two waves s1(t) and s2(t), s2(t) =−a·s1(t). The number, combination, and arrangement order of pulses or sequences in the aforementioned contrast pulse sequence can be adjusted. Part or all of the nonlinear imaging sequences can be selected; any one or more nonlinear imaging sequences and one or more nonlinear imaging sequences can constitute a contrast pulse method capable of exciting microbubble nonlinearity.

In another embodiment, nonlinear imaging is performed by transmitting pulses to the imaging area in an alternating element manner. Optionally, the nonlinear imaging sequence includes multiple identical pulse signals. When transmitting the contrast pulse sequence to the imaging area, it also includes: dividing ultrasound array elements into multiple groups and transmitting the multiple identical pulse signals to the imaging area in an alternating manner through the multiple groups. For example: the contrast pulse sequence contains three sequences s1(t), s2(t), and s3(t), where s2(t) and s3(t) are identical to s1(t) in waveform. When sending s2(t), only half of the odd-numbered elements are activated, and when sending s3(t), half of the even-numbered elements are activated. For example, with 128 elements, the odd group is 1, 3, 5 . . . 127, and the even group is 2, 4, 6 . . . 128. Another example is dividing the elements into several groups, such as four groups, and transmitting alternately.

Then, proceed to step 103, where nonlinear filtering processing and beamforming are performed sequentially on each group of echo signals in the echo signal group sequence to obtain the corresponding nonlinear ultrasound image sequence.

Wherein, considering that the linear components of tissue signals are mainly concentrated around the fundamental frequency, when performing nonlinear filtering processing on echo signals, this embodiment finds linear deviation coefficients between a group of echoes by matching the fundamental frequency and its nearby components, and applies the obtained linear deviation coefficients to the corresponding echo signals to obtain only the nonlinear echo components retained from microbubbles. The finding linear deviation coefficients between a group of echoes by matching the fundamental frequency and its nearby components is further implemented by the following steps:

I. Perform Fourier transform on each group of echo signals constituting the contrast multi-pulse imaging strategy:

P₁[ω]=Σₙ₌₀^{N}p₁[n]e^{-jωn}, P₁[ω]=Σₙ₌₀^{N}p₂[n]e^{-jωn}, where p1[n] and p2[n] are the echo signals after two ultrasound transmissions respectively, w is the discrete frequency, n is the discrete time, and N is the number of sampling points for each reception;

II. Extract the fundamental frequency component and its nearby components after the above signal transformation:

P′₁[ω]=P₁[ω]|_{ωϵ[ω}_{₀}_{-Δω,ω}_{₀}_{+Δω]}, P′₂[ω]=P₂[ω]|_{ωϵ[ω}_{₀}_{-Δω,ω}_{₀}_{+Δω]}, where ω₀ is the fundamental frequency during transmission and reception, and ωΔ is the half bandwidth, which is related to the actual transmitted signal waveform;

III. To eliminate the linear component deviation, the difference between P′₁[ω] and P′₂[ω] should be minimized. For example, using the least squares method or gradient descent method to calculate the fundamental wave amplitude correction coefficient θ_{ω}_{₀} that minimizes the difference Σ_{ω}(P′₁[ω]−θ_{ω}_{₀}P′₂[ω])² between P′₁[ω] and P′₂[ω], and applying this fundamental wave amplitude correction coefficient ↓_{ω}_{₀} to p_{z}[n] to obtain p₂[n]=θ_{ω}_{₀}[n], which is recorded as the echo signal after fundamental wave amplitude correction of p₂[n], where ωₛ is the maximum sampling frequency. The purpose of the fundamental wave amplitude correction coefficient θ_{ω}_{₀} is to compensate for the amplitude deviation of the fundamental wave component in the echo signal during multiple measurements, thereby helping to suppress the fundamental wave component and accurately extract the nonlinear component. According to the characteristics of different contrast multi-pulse imaging strategies, the linear components in the echo can be completely canceled by performing arithmetic operations on p₁[n] and p′₂[n], retaining only the nonlinear echo components of microbubbles, thus achieving the effect of nonlinear filtering, for example:
Pulse inversion: p_{PI}[n]=p′₂[n]+p′₁[n];
Amplitude modulation: p_{AM}[n]=p′₂ [n]−a·p′₁[n];
Amplitude-phase modulation: p_{AMPI}[n]=p′₂ [n]+a·p′₁[n];
Odd-even alternation: p_{OE} [n]=p′₃[n]−(p′₁[n]+p′₂[n])′.

For the sake of concise formula description, the above P₁[ω] and P₂[ω] both refer to echo signals that have been subjected to corresponding amplitude and phase basic correction processing according to the principles of different contrast multi-pulse imaging strategies, completely canceling the linear components of tissue signals and retaining only the nonlinear echo components of microbubbles according to the principles of different contrast multi-pulse imaging strategies. It can be understood that although this embodiment only lists the use of the least squares method or gradient descent method to calculate the linear deviation coefficient, this invention is not limited to the least squares method or gradient descent method, and equivalent or similar methods that can achieve the above process are within the scope of protection of this invention.

Then, proceed to step 104, identify and locate microbubbles in each frame of the nonlinear ultrasound image sequence frame by frame, and track microbubble trajectories based on the identification and localization results, where one microbubble trajectory is determined by the identification and localization results of microbubbles in N consecutive frames in the image sequence.

Then, proceed to step 105, reconstruct a super-resolution microvascular blood flow image based on the tracked microbubble trajectories. Optionally, the reconstructed microvascular blood flow image can be obtained through blood flow vector imaging, for example, by vectorizing the position and velocity of microbubbles to obtain a blood flow vector image.

Among them, steps 103 to 105 above perform nonlinear filtering processing and beamforming on the echo signals, and perform microbubble tracking on the synthesized nonlinear ultrasound images to obtain “low flow velocity” microvascular blood flow information. In order to obtain microvascular blood flow information for the entire velocity range, in one example, steps 103 to 105 may further include: performing linear filtering processing and beamforming on each group of echo signals in the echo signal group sequence in turn, as well as performing nonlinear filtering processing and beamforming on each group of echo signals in the echo signal group sequence in turn, to obtain corresponding linear ultrasound image sequences and nonlinear ultrasound image sequences; identifying and locating microbubbles in each frame of the linear ultrasound image sequence and the nonlinear ultrasound image sequence respectively, tracking microbubble trajectories based on the identification and localization results, and determining duplicated microbubble trajectories in time-aligned linear ultrasound images and nonlinear ultrasound images between the two image sequences and integrating them into a new trajectory; and reconstructing a super-resolution microvascular blood flow image based on the tracked microbubble trajectories and the integrated new trajectories. It can be understood that this example performs linear and nonlinear filtering processing on the echo signals respectively and synthesizes linear and nonlinear ultrasound images respectively, and performs microbubble tracking on linear and nonlinear ultrasound images respectively, and then reconstructs microvascular blood flow images based on the microbubble tracking results of the two images. Considering that there may be duplicate microbubble tracking results between the two images that affect the reconstruction results, before reconstruction, by determining duplicated microbubble trajectories in time-aligned linear ultrasound images and nonlinear ultrasound images between the two image sequences and integrating them into a new trajectory, the final full-velocity range microvascular blood flow information can be obtained.

Furthermore, the aforementioned “determining duplicated microbubble trajectories in time-aligned linear ultrasound images and nonlinear ultrasound images between the two image sequences and integrating them into a new trajectory” can be implemented through the following steps a and b: step a, calculating the corresponding velocity based on the tracking results of each microbubble trajectory; step b, if the absolute value of the velocity difference between two trajectories in time-aligned linear ultrasound images and nonlinear ultrasound images between the two image sequences is less than a first predetermined threshold, and the average value of the Euclidean distances of the point-by-point positions of these two trajectories is less than a second predetermined threshold, then these two trajectories are determined as the duplicated microbubble trajectories and integrated into a new trajectory. After obtaining the microbubble trajectories, the corresponding microbubble velocity is calculated based on the microbubble displacement and time interval between frames.

Optionally, the sampling frequency for transmitting the contrast pulse sequence and receiving echoes may include, but is not limited to, the Nyquist frequency (i.e., >2 times the highest frequency of the signal). Specifically, when the sampling frequency is set to more than 2 times the center frequency of the probe, nonlinear high-order harmonic signals (n times the excitation center frequency, n>2) from the echo of the forced vibration of the microbubbles can be received; when the sampling frequency is set to 2 times the center frequency of the probe, nonlinear subharmonic signals (lower than the excitation center frequency, such as ⅓, ½, ⅔, etc.) from the echoes of the forced vibration of the microbubbles, as well as the nonlinear signal components within the excitation bandwidth extracted by various contrast pulse imaging strategies mentioned above due to nonlinearity, can be received.

### Second Embodiment

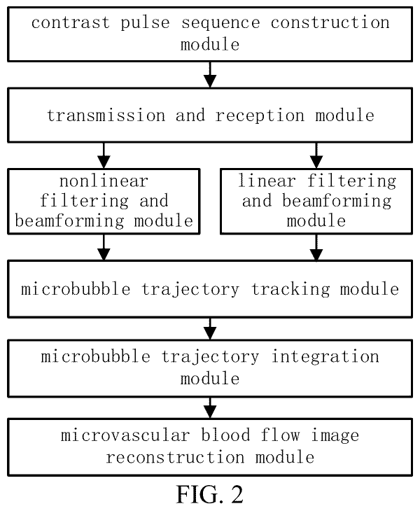

The second embodiment of this invention relates to a microvascular blood flow ultrasound imaging system, the structure of which is shown in FIG. 2. The microvascular blood flow ultrasound imaging system includes a contrast pulse sequence construction module, a transmission and reception module, a nonlinear filtering and beamforming module, a microbubble trajectory tracking module, and a microvascular blood flow image reconstruction module.

The contrast pulse sequence construction module is configured to construct a contrast pulse sequence containing linear imaging sequences and nonlinear imaging sequences.

The transmission and reception module is configured to transmit the contrast pulse sequence to the imaging area and obtain multiple groups of echo signals within a preset time period to form an echo signal group sequence, wherein ultrasound microbubbles are injected into the blood vessels of the imaging area.

The nonlinear filtering and beamforming module is configured to sequentially perform nonlinear filtering processing and beamforming on each group of echo signals in the echo signal group sequence to obtain a corresponding nonlinear ultrasound image sequence.

Optionally, the system also includes a linear filtering and beamforming module, which is configured to sequentially perform linear filtering processing and beamforming on each group of echo signals in the echo signal group sequence to obtain a corresponding linear ultrasound image sequence. The microbubble trajectory tracking module is configured to identify and locate microbubbles in each frame of the nonlinear ultrasound image sequence frame by frame, and track microbubble trajectories based on the identification and localization results, wherein one microbubble trajectory is determined by the identification and localization results of microbubbles in N consecutive frames in the image sequence. The microvascular blood flow image reconstruction module is configured to reconstruct a super-resolution microvascular blood flow image based on the tracked microbubble trajectories.

In one embodiment, the microbubble trajectory tracking module is also configured to identify and locate microbubbles in each frame of both the linear ultrasound image sequence and the nonlinear ultrasound image sequence frame by frame, and track microbubble trajectories based on the identification and localization results. In this embodiment, the system also includes a microbubble trajectory integration module, which is configured to identify duplicated microbubble trajectories in time-aligned linear ultrasound images and nonlinear ultrasound images from the two image sequences and integrate them into a new trajectory. The microvascular blood flow image reconstruction module is also configured to reconstruct a super-resolution microvascular blood flow image based on the tracked microbubble trajectories and the integrated new trajectories.

Optionally, the microbubble trajectory integration module is also configured to calculate the corresponding velocity for each microbubble trajectory based on its tracking results. If the absolute value of the velocity difference between two trajectories in time-aligned linear ultrasound images and nonlinear ultrasound images from the two image sequences is less than a first predetermined threshold, and the average Euclidean distance of the point-by-point positions of these two trajectories is less than a second predetermined threshold, then these two trajectories are determined to be the duplicated microbubble trajectories and integrated into a new trajectory.

It should be noted that the first embodiment is a corresponding method embodiment to this embodiment. The technical details in the first embodiment can be applied to this embodiment, and the technical details in this embodiment can also be applied to the first embodiment.

### Third Embodiment

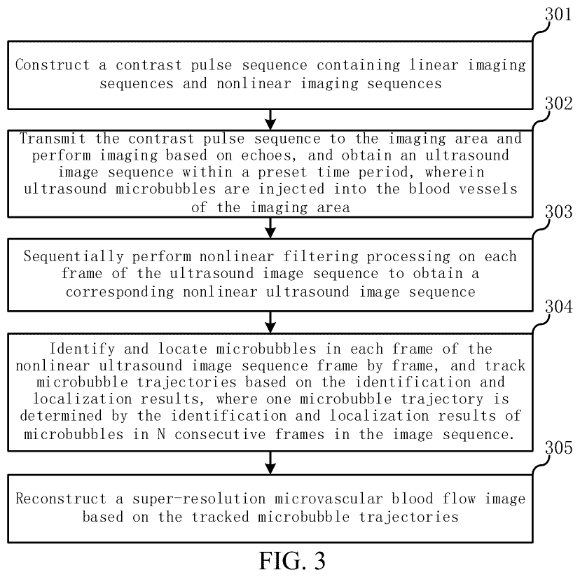

The third embodiment of this invention relates to a microvascular blood flow ultrasound imaging method, the process of which is shown in FIG. 3. The method includes the following steps:

In step 301, construct a contrast pulse sequence containing linear imaging sequences and nonlinear imaging sequences. Then, proceed to step 302, transmit the contrast pulse sequence to the imaging area and perform imaging based on echoes, and obtain an ultrasound image sequence within a preset time period, wherein ultrasound microbubbles are injected into the blood vessels of the imaging area. Optionally, in step 302, plane wave imaging, focused wave imaging, diverging wave imaging, synthetic aperture imaging, etc., can be performed on the imaging area using the contrast pulse sequence, but not limited to these.

In one embodiment, nonlinear contrast pulses are obtained by modulating linear waves, and nonlinear imaging is performed by transmitting a contrast pulse sequence containing these contrast pulses to the imaging area. Optionally, the nonlinear imaging sequence includes linear sequence and modulation sequence pairs, where the modulation sequence in the linear sequence and modulation sequence pair is obtained by performing a preset modulation method on the linear sequence. The preset modulation method includes one or more of the following: pulse inversion, amplitude modulation, and amplitude-phase modulation. Among them, the pulse inversion includes two sequences s1(t) and s2(t), where s2(t)=−s1(t); the amplitude modulation includes two waves s1(t) and s2(t), where s2(t)=a·s1(t); the amplitude-phase modulation includes two waves s1(t) and s2(t), where s2(t) =−a·s1(t). The number, combination, and arrangement order of pulses or sequences in the aforementioned contrast pulse sequence can be adjusted. Part or all of the nonlinear imaging sequences can be selected; any one or more nonlinear imaging sequences and one or more nonlinear imaging sequences can be selected to form a contrast pulse method capable of exciting microbubble nonlinearity.

In another embodiment, nonlinear imaging is performed by transmitting pulses to the imaging area using alternating array elements. Optionally, the nonlinear imaging sequence includes multiple identical pulse signals. When transmitting the contrast pulse sequence to the imaging area, it also includes: dividing the ultrasound array elements into multiple groups and transmitting the multiple identical pulse signals to the imaging area through alternating transmission of these multiple groups. For example, the contrast pulse sequence contains three sequences s1(t), s2(t), and s3(t), where s2(t) and s3(t) are identical to s1(t) in waveform. When transmitting s2(t), only half of the odd-numbered array elements are activated, and when transmitting s3(t), half of the even-numbered array elements are activated. For example, with 128 array elements, the odd group includes 1, 3, 5, . . . , 127, and the even group includes 2, 4, 6, . . . , 128. Another example is dividing the array elements into several groups, such as four groups, and transmitting alternately.

Then, proceed to step 303, sequentially perform nonlinear filtering processing on each frame of the ultrasound image sequence to obtain a corresponding nonlinear ultrasound image sequence. Considering that the linear components of tissue signals are mainly concentrated at the fundamental frequency and its vicinity, when performing nonlinear filtering processing on each frame of the ultrasound image sequence, this invention finds a set of linear deviation coefficients between echoes by matching the fundamental frequency and its nearby components, and applies the obtained linear deviation coefficients to the corresponding image to obtain only the nonlinear components of microbubbles. The finding linear deviation coefficients between a group of echoes by matching the fundamental frequency and its nearby components is further implemented by the following steps: For the echo signals obtained under the contrast multi-pulse imaging strategy and then beamforming B-mode images, perform Fourier transform along the axial direction respectively: M₁[ω]=Σₙ₌₀^{N}m₁[n]e^{-jω}, M₂[ω]=Σₙ₌₀^{N}m₂[n]e^{-jωn}, where m₁[n] and m₂[n] are the image column (axial) signals after two ultrasound wave transmissions respectively, w is a discrete frequency, n is a discrete time, and N is a number of sampling points for each reception;

Extract the fundamental frequency and nearby components M′₁[ω] and M′₂[ω] of the B-mode image column (axial) after Fourier transform: M₁[ω]=M₁[ω]|_{ωϵ[ω}_{₀}_{-Δω,ω}_{₀}_{+Δω]}, M′₂[ω]=M₂[ω]|_{ωϵ[w}_{₀}_{-Δω,ω}_{₀}_{+Δω]}, where ω₀ is the fundamental frequency during transmission and reception, Δωis the half bandwidth, related to the actual transmitted signal waveform;

Calculate the fundamental wave amplitude correction coefficient θ_{ω}_{₀} between the image columns (axial) that minimizes the difference Σ_{ω}(M′₁[ω]−θ_{ω}_{₀}M′₂[ω])² between M′₁[ω] and M′₂[ω] using the least squares method or gradient descent method, and apply this fundamental wave amplitude correction coefficient θ_{ω}_{₀} to m₂[n] to obtain m′₂[n]=θ_{ω}_{₀}m₂[n], which is recorded as the image column (axial) signal after fundamental wave amplitude correction of m₂[n], where ωₛ is the maximum sampling frequency. It should be noted that the purpose of the fundamental wave amplitude correction coefficient θ_{ω}_{₀} here is to compensate for the amplitude deviation of the fundamental wave component in the image column (axial) signal during multiple measurements, thereby contributing to the suppression of the fundamental wave component and accurately extracting the nonlinear component. According to the characteristics of different contrast multi-pulse imaging strategies, the linear components in the image can be completely canceled by performing arithmetic operations on m₁[n] and m′₂[n], retaining only the nonlinear echo components of microbubbles, achieving the effect of nonlinear filtering.

Finally, apply the obtained linear deviation coefficient to the corresponding echo signal and perform an inverse Fourier transform to obtain only the nonlinear echo components of microbubbles. It can be understood that although this embodiment only lists the use of the least squares method or gradient descent method to calculate the linear deviation coefficient, this invention is not limited to the least squares method or gradient descent method. Equivalent or similar methods that can achieve the above process are within the scope of protection of this invention.

Then, proceed to step 304, identify and locate microbubbles in each frame of the nonlinear ultrasound image sequence frame by frame, and track microbubble trajectories based on the identification and localization results, where one microbubble trajectory is determined by the identification and localization results of microbubbles in N consecutive frames in the image sequence. Then, proceed to step 305, reconstruct a super-resolution microvascular blood flow image based on the tracked microbubble trajectories. Optionally, the reconstructed microvascular blood flow image can be obtained through blood flow vector imaging to obtain a blood flow vector image. Among them, steps 303 to 305 above perform nonlinear filtering processing on the imaging image and microbubble tracking on the nonlinear ultrasound image to obtain an image of “low flow velocity” microvascular blood flow information. In order to obtain an image of microvascular blood flow information for all velocity intervals, in one embodiment, steps 303 to 305 may further include: performing linear filtering processing on each frame of the ultrasound image sequence in sequence to obtain a corresponding linear ultrasound image sequence, and performing nonlinear filtering processing on each frame of the ultrasound image sequence in sequence to obtain a nonlinear ultrasound image sequence; identifying and locating microbubbles in each frame of the linear ultrasound image sequence and the nonlinear ultrasound image sequence respectively, and tracking microbubble trajectories based on the identification and localization results; determining duplicated microbubble trajectories in time-aligned linear ultrasound images and nonlinear ultrasound images between the two image sequences and integrating them into a new trajectory; reconstructing a super-resolution microvascular blood flow image based on the tracked microbubble trajectories and the integrated new trajectories.

Furthermore, the aforementioned “determining duplicated microbubble trajectories in time-aligned linear ultrasound images and nonlinear ultrasound images between the two image sequences and integrating them into a new trajectory, where a microbubble trajectory is determined by the identification and localization results of microbubbles in N consecutive frames in the image sequence” can be implemented through the following steps A and B: Step A, calculate the corresponding velocity based on the tracking results of each microbubble trajectory; Step B, if the absolute value of the velocity difference between two trajectories in time-aligned linear ultrasound images and nonlinear ultrasound images between the two image sequences is less than a first predetermined threshold, and the average value of the Euclidean distances of the point-by-point positions of these two trajectories is less than a second predetermined threshold, then determine these two trajectories as the duplicated microbubble trajectories and integrate them into a new trajectory.

Optionally, the sampling frequency for transmitting the contrast pulse sequence and receiving echoes may include, but is not limited to, the Nyquist frequency (i.e., >2 times the highest frequency of the signal). Specifically, when the sampling frequency is set to more than 2 times the center frequency of the probe, nonlinear high-order harmonic signals (n times the excitation center frequency, n>2) from the echo of the forced vibration of the microbubbles can be received; when the sampling frequency is set to 2 times the center frequency of the probe, nonlinear subharmonic signals (lower than the excitation center frequency, such as ⅓, ½, ⅔, etc.) from the echoes of the forced vibration of the microbubbles, as well as the nonlinear signal components within the excitation bandwidth extracted by various contrast pulse imaging strategies mentioned above due to nonlinearity, can be received.

### Fourth Embodiment

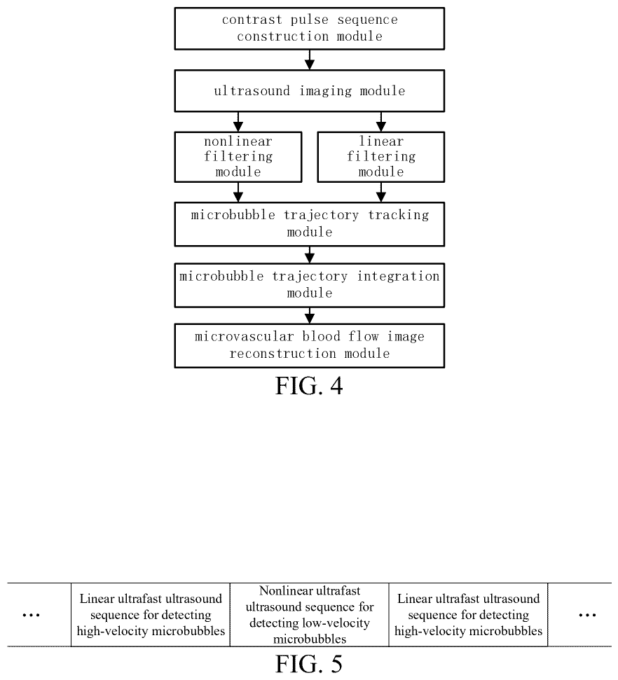

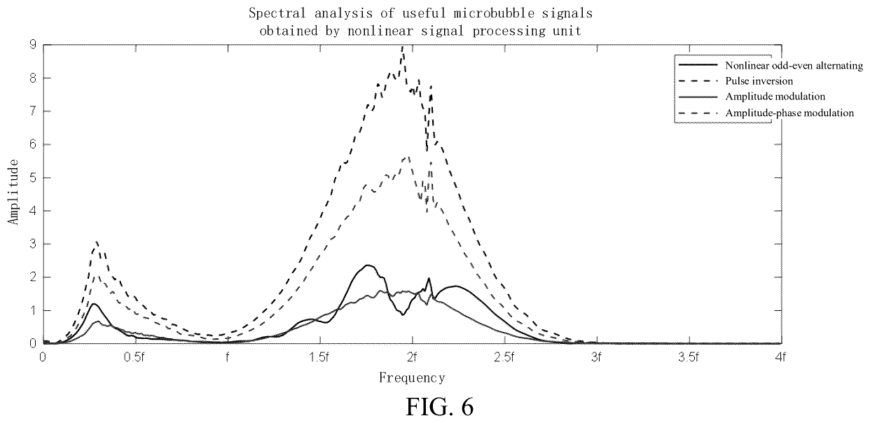

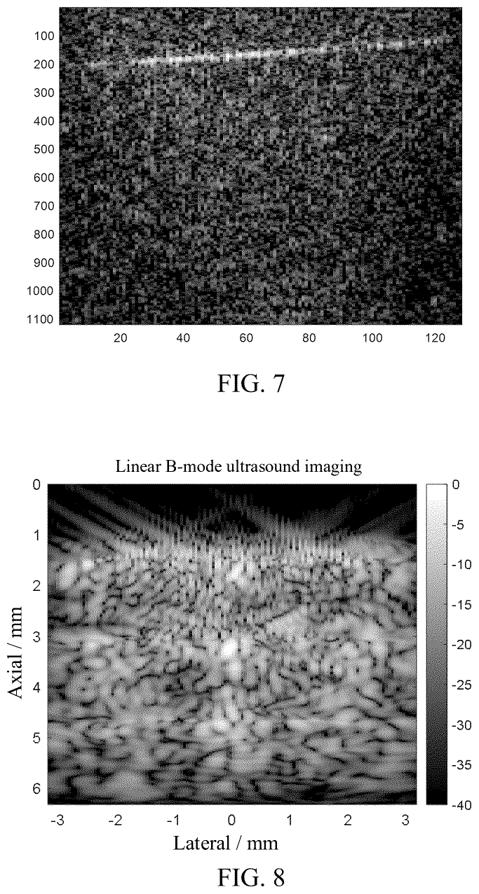

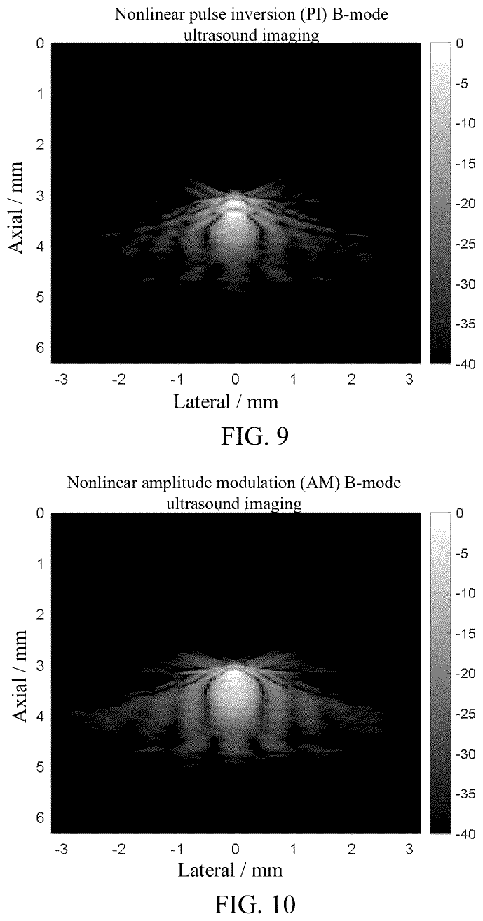

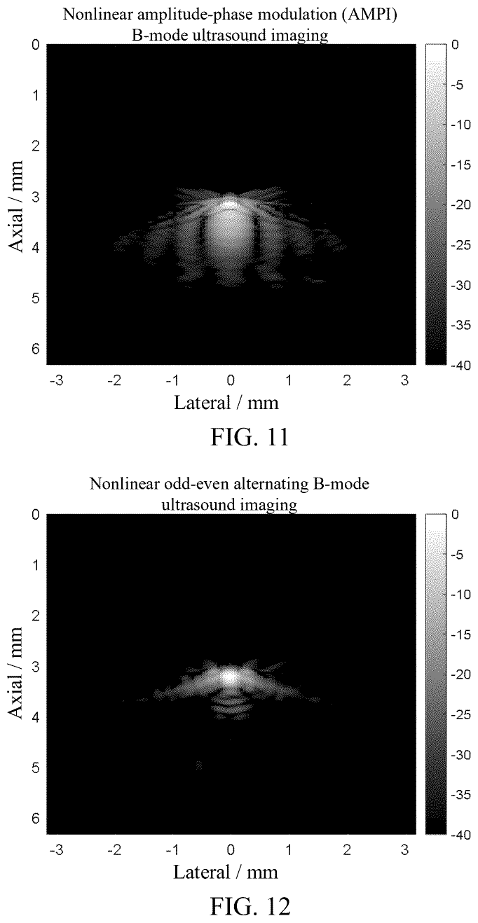

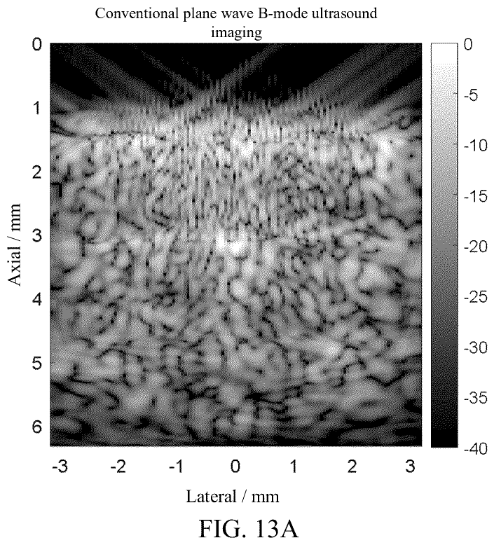

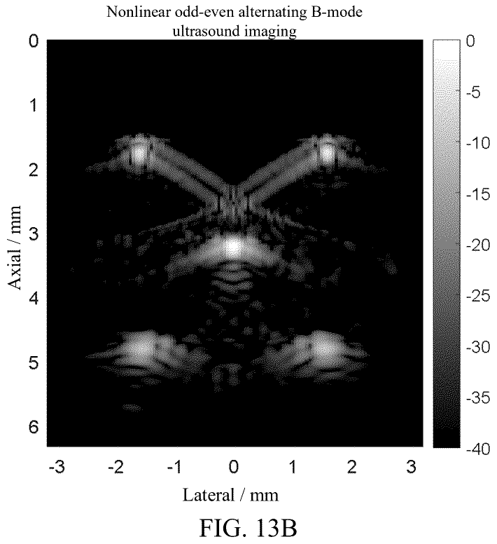

The fourth embodiment of this invention relates to a microvascular blood flow ultrasound imaging system, the structure of which is shown in FIG. 4. The microvascular blood flow ultrasound imaging system includes a contrast pulse sequence construction module, an ultrasound imaging module, a nonlinear filtering module, a microbubble trajectory tracking module, and a microvascular blood flow image reconstruction module.

Specifically, the contrast pulse sequence construction module is used to construct a contrast pulse sequence containing linear imaging sequences and nonlinear imaging sequences.

The ultrasound imaging module is used to transmit the contrast pulse sequence to the imaging area and perform imaging based on echoes, and obtain an ultrasound image sequence within a preset time period, where ultrasound microbubbles are injected into the blood vessels of the imaging area.

The nonlinear filtering module is used to perform nonlinear filtering processing on each frame of the ultrasound image sequence in sequence to obtain a corresponding nonlinear ultrasound image sequence.

The microbubble trajectory tracking module is used to identify and locate microbubbles in each frame of the nonlinear ultrasound image sequence frame by frame, and track microbubble trajectories based on the identification and localization results, where a microbubble trajectory is determined by the identification and localization results of microbubbles in N consecutive frames in the image sequence. The microvascular blood flow image reconstruction module is used to reconstruct a super-resolution microvascular blood flow image based on the tracked microbubble trajectories.

In one embodiment, the system further includes a linear filtering module and a microbubble trajectory integration module. The linear filtering module is configured to perform linear filtering processing on each frame of the ultrasound image sequence in sequence to obtain a corresponding linear ultrasound image sequence. In this embodiment, the microbubble trajectory tracking module is further configured to identify and locate microbubbles in each frame of the linear ultrasound image sequence and the nonlinear ultrasound image sequence respectively, frame by frame, and track microbubble trajectories based on the identification and localization results. The microbubble trajectory integration module is configured to determine duplicated microbubble trajectories in time-aligned linear ultrasound images and nonlinear ultrasound images from the two image sequences and integrate them into a new trajectory. The microvascular blood flow image reconstruction module is further configured to reconstruct a super-resolution microvascular blood flow image based on the tracked microbubble trajectories and the integrated new trajectories.

Optionally, the microbubble trajectory integration module is further configured to calculate corresponding velocities based on the tracking results of each microbubble trajectory. If the absolute value of the velocity difference between two trajectories in time-aligned linear ultrasound images and nonlinear ultrasound images from the two image sequences is less than a first predetermined threshold, and the average Euclidean distance of the point-by-point positions of these two trajectories is less than a second predetermined threshold, then these two trajectories are determined to be the duplicated microbubble trajectories and integrated into a new trajectory.

It should be noted that the third embodiment is the corresponding method embodiment of this embodiment. The technical details in the third embodiment can be applied to this embodiment, and the technical details in this embodiment can also be applied to the third embodiment.

To better understand the technical solution of this invention, the embodiments of this invention will be illustrated by applying them to four examples (Examples 1 to 4) of rat spinal cord. These examples are ultrasound plane wave simulation imaging processes using the super-resolution ultrasound microvascular imaging system based on nonlinear ultrasound in this invention, but this invention is not limited to simulation imaging.

### Example 1

(1) Design a linear and nonlinear combined ultrafast ultrasound contrast pulse imaging sequence. The constructed contrast pulse sequence S(t) sequentially includes: linear sequence s1(t), nonlinear sequence s2(t), linear sequence s3(t); where s1(t)=s3(t), s2(t) =−s1(t).

(2) Perform ultrasound plane wave simulation using the k-Waves tool. Use the linear and nonlinear combined ultrafast ultrasound contrast pulse sequence described in (1) to perform ultrasound plane wave scanning on the imaging area, with simulated slowly moving microbubbles in the area, to obtain 20 frames of ultrasound echo signals at a frame rate of 500 Hz and a microbubble velocity of about 1 mm/s. The specific simulation steps are as follows:
(2-1) perform plane wave imaging using the contrast pulse sequence, without considering the existence of microbubbles, only generating random background noise in the imaging area; receive echo signals n1(t) to n5(t);
(2-2) in the imaging process of (2-1), assume the position where microbubbles exist, and record the pressure field information p1(t) to p5(t) at that position;
(2-3) use the pressure field information p1(t) to p5(t) as input to the Rayleigh-Plesset equation to obtain the forced vibration responses pr1(t) to pr5(t) of the microbubbles;
(2-4) simulate an excitation source at the assumed microbubble position, transmit the microbubble responses pr1(t) to pr5(t) as source signals into the imaging area, and receive echo signals m1(t) to m5(t) by an ultrasound probe;
(2-5) add the echo signals n1(t) to n5(t) from (2-1) and the echo signals m1(t) to m5(t) from (2-4) correspondingly to obtain complete plane wave ultrasound simulation echo signals RF1(t) to RF5(t);
(2-6) slowly move the position of the microbubble, perform the next frame of plane wave ultrasound simulation, repeat (2-1) to (2-5); collect a total of 50 frames.

(3) Apply singular value decomposition (SVD) filter to remove clutter from linear echo signals RF1(t), RF3(t), RF5(t).

(4) Extract useful signals based on microbubble nonlinearity from nonlinear echo signals RF2(t), RF4(t). The specific extraction steps are as follows:
(4-1) Calculate linear deviation coefficients k1 and k2 to minimize the L1 norm;
(4-2) RF1(t) and RF2(t) form a pair of pulse inversion contrast sequence method echo signals, cancel the linear component of background noise echoes, and obtain useful microbubble nonlinear response echo RF_PI(t)=RF1(t)+k1·RF2(t).

(5) Perform beamforming to obtain plane wave ultrasound images of linear echoes and nonlinear echoes respectively.

(6) Accurately locate microbubble positions in each frame of linear images and nonlinear images respectively.

(7) Summarize the microbubble positions obtained from linear images and nonlinear images, complete microbubble pairing and trajectory generation between frames, and obtain the final super-resolution ultrasound imaging results.

### Example 2

(1) Design a linear and nonlinear combined ultrafast ultrasound contrast pulse imaging sequence. The constructed contrast pulse sequence S(t) sequentially includes: linear sequence s1(t), nonlinear sequence s2(t), linear sequence s3(t), nonlinear sequence s4(t), linear sequence s5(t); where s1(t)=s2(t)=s3(t)=s4(t)=s5(t), note that only odd-numbered array elements are activated when transmitting s2(t), and the remaining even-numbered array elements are activated when transmitting s4(t).

(2) Perform ultrasound plane wave simulation using the k-Waves tool. Use the linear and nonlinear combined ultrafast ultrasound contrast pulse sequence described in (1) to perform ultrasound plane wave scanning on the imaging area, with simulated slowly moving microbubbles in the area, to obtain 50 frames of ultrasound echo signals at a frame rate of 500 Hz and a microbubble velocity of about 1 mm/s. The specific simulation steps are as follows:
(2-1) perform plane wave imaging using the contrast pulse sequence, without considering the existence of microbubbles, only generating random background noise in the imaging area; receive echo signals n1(t) to n5(t);
(2-2) in the imaging process of (2-1), assume the position where microbubbles exist, and record the pressure field information p1(t) to p5 (t) at that position;
(2-3) use the pressure field information p1(t) to p5(t) as input to the Rayleigh-Plesset equation to obtain the forced vibration responses pr1(t) to pr5(t) of the microbubbles;
(2-4) simulate an excitation source at the assumed microbubble position, transmit the microbubble responses pr1(t) to pr5(t) as source signals into the imaging area, and receive echo signals m1(t) to m5(t) by an ultrasound probe;
(2-5) add the echo signals n1(t) to n5(t) from (2-1) and the echo signals m1(t) to m5(t) from (2-4) correspondingly to obtain complete plane wave ultrasound simulation echo signals RF1(t) to RF5(t);
(2-6) slowly move the position of the microbubble, perform the next frame of plane wave ultrasound simulation, repeat (2-1) to (2-5); collect a total of 50 frames.

(3) apply singular value decomposition (SVD) filter to remove clutter from linear echo signals RF1(t), RF3(t), RF5(t).

(4) extract useful signals based on microbubble nonlinearity from nonlinear echo signals RF2(t), RF4(t). The specific extraction steps are as follows:
(4-1) Calculate linear deviation coefficients k1 and k2 to minimize the L2 norm;
(4-2) RF1(t), RF2(t), and RF4(t) constitute a group of odd-even alternating contrast sequence method echo signals, canceling the linear component of the background noise echo to obtain the useful microbubble nonlinear response echo RF_OE1(t)=RF1(t)−k1·(RF2(t)+RF4(t));
(4-3) RF3(t), RF2(t), and RF4(t) constitute another group of odd-even alternating contrast sequence method echo signals, canceling the linear component of the background noise echo to obtain the useful microbubble nonlinear response echo RF_OE2(t)=RF3(t)−k2. (RF2(t)+RF4(t)).

(5) Beamforming is performed to obtain plane wave ultrasound images of linear echoes and nonlinear echoes respectively.

(6) Precise localization of microbubble positions is performed for each frame of linear and nonlinear images respectively.

(7) The microbubble positions obtained from linear and nonlinear images are aggregated, and microbubble pairing and trajectory generation are completed between frames to obtain the final super-resolution ultrasound imaging result.

### Example 3

(1) Design a linear and nonlinear combined ultrafast ultrasound contrast pulse imaging sequence. The constructed contrast pulse sequence S(t) sequentially includes: linear sequence s1(t), nonlinear sequence s2(t), linear sequence s3(t); where s1(t)=s3(t), s2(t)=2*s1(t).

(2) Perform ultrasound plane wave simulation using the k-Waves tool. Use the linear and nonlinear combined ultrafast ultrasound contrast pulse sequence described in (1) to perform ultrasound plane wave scanning of the imaging area, with simulated slowly moving microbubbles in the area, to obtain 20 frames of ultrasound echo signals at a frame rate of 500 Hz and a microbubble velocity of approximately 2 mm/s. The specific simulation steps are as follows:
(2-1) perform plane wave imaging using the contrast pulse sequence, without considering the existence of microbubbles, only generating random background noise in the imaging area; receive echo signals n1(t) to n5(t);
(2-2) in the imaging process of (2-1), assume the position where microbubbles exist and record the pressure field information p1(t) to p5(t) at that position;
(2-3) use the pressure field information p1(t) to p5(t) as input for the Rayleigh-Plesset equation to obtain the forced vibration responses pr1(t) to pr5(t) of the microbubbles;
(2-4) simulate an excitation source at the assumed microbubble position, transmit the microbubble responses pr1(t) to pr5(t) as source signals into the imaging area, and receive echo signals m1(t) to m5(t) by an ultrasound probe;
(2-5) add the corresponding echo signals n1(t) to n5(t) from (2-1) and m1(t) to m5(t) from (2-4) to obtain the complete plane wave ultrasound simulation echo signals RF1(t) to RF5(t);

(2-6) slowly move the microbubble position and perform the next frame of plane wave ultrasound simulation, repeating steps (2-1) to (2-5); collect a total of 50 frames.

(3) Apply singular value decomposition (SVD) filter to remove clutter from the linear echo signals RF1(t), RF3(t), and RF5(t).

(4) Extract useful signals based on microbubble nonlinearity from the nonlinear echo signals RF2(t) and RF4(t). The specific extraction steps are as follows:
(4-1) Calculate the linear deviation coefficients k1 and k2 to minimize the L1 norm;
(4-2) RF1(t) and RF2(t) form a pair of amplitude modulation contrast sequence method echo signals, canceling the linear component of the background noise echo to obtain the useful microbubble nonlinear response echo RF_PI(t)=RF1(t)−0.5·k1·RF2(t).

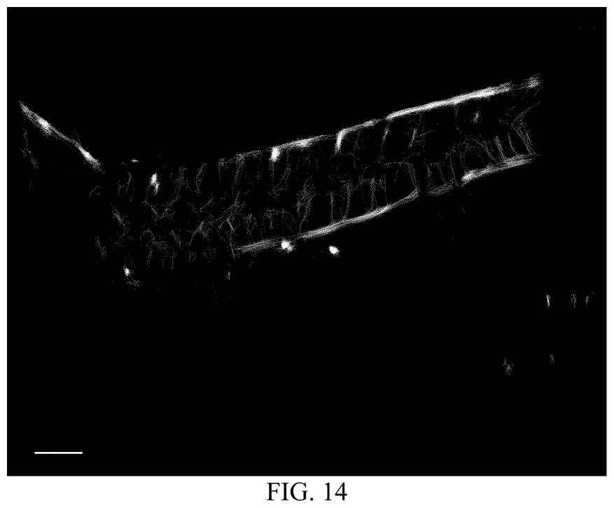

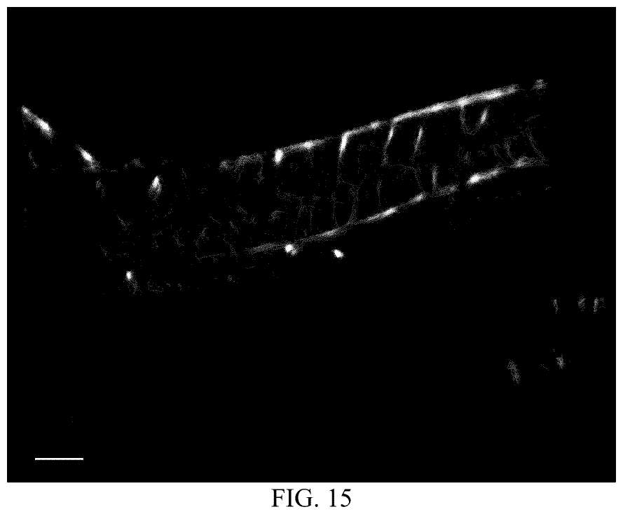

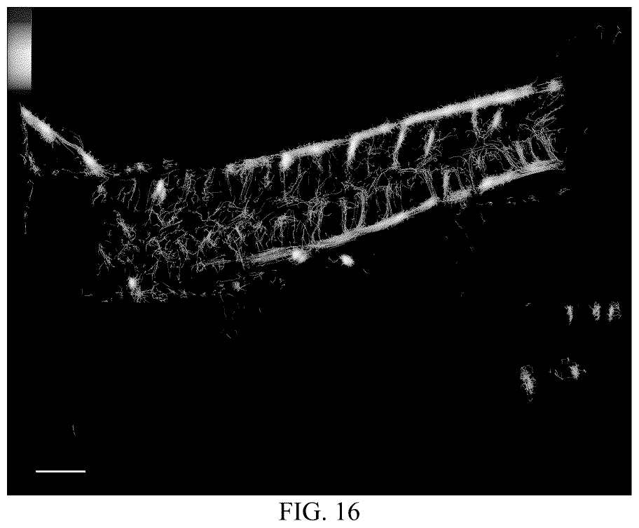

(5) Perform beamforming to obtain plane wave ultrasound images of linear echoes (as shown in FIG. 14) and nonlinear echoes (as shown in FIG. 15) respectively.

(6) Precisely localize microbubble positions in each frame of linear and nonlinear images respectively.

(7) Aggregate the microbubble positions obtained from linear and nonlinear images, and complete microbubble pairing and trajectory generation between frames to obtain the final super-resolution ultrasound imaging result (as shown in FIG. 16).

### Example 4

(1) Design a linear and nonlinear combined ultrafast ultrasound contrast pulse imaging sequence. The constructed contrast pulse sequence S(t) sequentially includes: linear sequence s1(t), nonlinear sequence s2(t), linear sequence s3(t), nonlinear sequence s4(t), linear sequence s5(t); where s1(t)=s3(t)=s5(t), s2(t)=−s1(t), s4(t)=2*s3(t).

(2) Perform ultrasound plane wave simulation using the k-Waves tool. Use the linear and nonlinear combined ultrafast ultrasound contrast pulse sequence described in (1) to perform ultrasound plane wave scanning of the imaging area.

(3) Apply singular value decomposition (SVD) filter to remove clutter from the linear echo signals RF1(t), RF3(t), and RF5(t).

(4) Extract useful signals based on microbubble nonlinearity from the nonlinear echo signals RF2(t) and RF4(t). The specific extraction steps are as follows:
(4-1) Calculate the linear deviation coefficients k1 and k2 to minimize the L2 norm;
(4-2) RF1(t) and RF2(t) form a pair of pulse inversion contrast sequence method echo signals, canceling the linear component of the background noise echo to obtain the useful microbubble nonlinear response echo RF_PI(t)=RF1(t)+k1·RF2(t);
(4-3) RF3(t) and RF4(t) form a pair of amplitude modulation contrast sequence method echo signals, canceling the linear component of the background noise echo to obtain the useful microbubble nonlinear response echo RF_AM(t)=RF3(t)−0.5·k2·RF2(t).
(5) Perform beamforming to obtain plane wave ultrasound images of linear echoes and nonlinear echoes respectively.
(6) Precisely localize microbubble positions in each frame of linear and nonlinear images respectively.
(7) Perform trajectory tracking and calculate vectorized velocity information for microbubble positions obtained from linear filtering method and nonlinear filtering method separately; to avoid duplicate blood flow information, find all microbubble trajectories extracted by both linear filtering method and nonlinear filtering method, and integrate each pair of (two) duplicate trajectories into one new trajectory, namely:
(i) Save the microbubble trajectories and vectorized velocity information obtained from linear filtering method and nonlinear filtering method separately, search for and mark all trajectories with similar velocities between the two groups (e.g., velocity difference within +0.2 mm/s).
(ii) For the groups of trajectories with similar velocities recorded in (i), further check if the positions of the two groups of trajectories are close, using a point-by-point comparison method to judge the positions.
(iii) For the trajectories with both velocity and position information found to be close in (ii), merge the information of the two groups of trajectories into one, where the number of microbubble position points is taken as the larger number of points from the two groups of trajectories, replacing individual microbubble position points to make the trajectory more statistically significant.

(8) Superimpose the microbubble trajectory information obtained from linear filtering method and nonlinear filtering method to obtain microbubble trajectories containing all velocity intervals, and reconstruct the super-resolution microvascular blood flow image.

It should be noted that those skilled in the art should understand that the implementation functions of the various modules shown in the above-mentioned embodiments of the microvascular blood flow ultrasound imaging system can be understood with reference to the relevant descriptions of the corresponding aforementioned microvascular blood flow ultrasound imaging method. The functions of the various modules shown in the above-mentioned embodiments of the microvascular blood flow ultrasound imaging system can be realized by programs (executable instructions) running on a processor, or can be realized by specific logic circuits. When the above-mentioned microvascular blood flow ultrasound imaging system of the embodiments of the present invention is implemented in the form of software functional modules and sold or used as an independent product, it can also be stored in a computer-readable storage medium. Based on this understanding, the technical solution of the embodiments of the present invention or the part that contributes to the prior art can be embodied in the form of a software product, which is stored in a storage medium and includes several instructions for enabling a computer device (which may be a personal computer, server, or network device, etc.) to perform all or part of the methods described in the various embodiments of the present invention. The aforementioned storage medium includes: U disk, mobile hard disk, read-only memory (ROM), magnetic disk or optical disk, and various media that can store program codes. In this way, the embodiments of the present invention are not limited to any specific combination of hardware and software.

Correspondingly, the embodiments of the present invention also provide a computer-readable storage medium, which stores computer executable instructions, and when the computer executable instructions are executed by a processor, the methods of the various embodiments of the present invention are implemented. The computer-readable storage medium includes permanent and non-permanent, removable and non-removable media that can be implemented by any method or technology for information storage. Information can be computer-readable instructions, data structures, program modules or other data. Examples of computer storage media include, but are not limited to, phase change memory (PRAM), static random access memory (SRAM), dynamic random access memory (DRAM), other types of random access memory (RAM), read-only memory (ROM), electrically erasable programmable read-only memory (EEPROM), flash memory or other memory technology, compact disc read-only memory (CD-ROM), digital versatile disc (DVD) or other optical storage, magnetic cassette tape, magnetic tape magnetic disk storage or other magnetic storage devices, or any other non-transmission medium that can be used to store information accessible by computing devices. As defined herein, computer-readable storage media do not include transitory media, such as modulated data signals and carriers.

In addition, the embodiments of the present invention also provide a microvascular blood flow ultrasound imaging system, which includes a memory for storing computer executable instructions, and a processor; the processor is used to implement the steps in the above-mentioned method embodiments when executing the computer executable instructions in the memory. The processor can be a central processing unit (CPU), and can also be other general-purpose processors, digital signal processors (DSP), invention-specific integrated circuits (ASIC), etc. The aforementioned memory can be read-only memory (ROM), random access memory (RAM), flash memory, hard disk or solid-state drive, etc. The steps of the method disclosed in the embodiments of the present invention may be directly embodied by a hardware processor, or may be implemented by a combination of hardware and software modules in a processor.

It should be noted that in the specification of the present invention, relational terms such as first, second, etc. are used only to distinguish one entity or operation from another entity or operation, and do not necessarily require or imply any actual relationship or sequence between such entities or operations. Moreover, the term “comprise”, “include”, or any other variants thereof are intended to cover a non-exclusive inclusion, such that a process, method, article, or apparatus that comprises a list of elements not only comprises those elements, but may also comprise other elements not expressly listed or inherent to such a process, method, article, or apparatus. Without further limitations, an element defined by the statement “comprising a” does not exclude the presence of additional identical elements in the process, method, article, or apparatus that comprises the element. In the specification of the present invention, if it is mentioned that an action is performed according to an element, it means that the action is performed at least according to the element, which includes two situations: (1) the behavior is performed only according to that element, and (2) the behavior is performed according to that element and other elements. The expressions ‘multiple’ and ‘a plurality of’ are defined to mean two or more than two.

All documents mentioned in this invention are deemed to be integrally included in the disclosure of this invention, so that they may serve as a basis for amendment if necessary. In addition, it shall be understood that the foregoing are merely better examples of the specification and are not intended to limit the scope of protection of the patent. Any modification, equivalent replacement, improvement, etc. within the spirit and principles of one or more embodiments of this specification shall be included in the scope of protection of such one or more embodiments of this specification.

1-22. (canceled)

23. A microvascular blood flow ultrasound imaging method, comprising:
constructing, by a processor, a contrast pulse sequence containing linear imaging sequences and nonlinear amplitude-phase excitation imaging sequences;
transmitting, by an ultrasound imaging device, the contrast pulse sequence to an imaging area, and acquiring multiple groups of echo signals within a preset time period to obtain filtered and beamforming linear ultrasound image sequences and nonlinear ultrasound image sequences, wherein blood vessels in the imaging area are injected with ultrasound microbubbles;
identifying and locating, by a processor, microbubbles frame by frame in each frame of the linear ultrasound image sequences and the nonlinear ultrasound image sequences respectively, and tracking microbubble trajectories based on identification and localization results, and determining and integrating duplicate microbubble trajectories in the time-aligned linear ultrasound images of the linear ultrasound image sequences and nonlinear ultrasound images of the nonlinear ultrasound image sequences into a new trajectory.
reconstructing, by a processor, a super-resolution microvascular blood flow image based on the tracked microbubble trajectories and integrated new trajectories.

24. The microvascular blood flow ultrasound imaging method of claim 23, wherein the acquiring multiple groups of echo signals within a preset time period to obtain filtered and beamforming linear ultrasound image sequences and nonlinear ultrasound image sequences, further comprises:
acquiring multiple groups of echo signals within a preset time period to form an echo signal group sequence;
using a linear filter to sequentially perform linear filtering processing and beamforming on each group of echo signals in the echo signal group sequence, and using a nonlinear filter to sequentially perform nonlinear filtering processing and beamforming on each group of echo signals in the echo signal group sequence, to obtain a corresponding linear ultrasound image sequence and nonlinear ultrasound image sequence.

25. The microvascular blood flow ultrasound imaging method of claim 24, wherein the determining and integrating duplicate microbubble trajectories in the time-aligned linear ultrasound images of the linear ultrasound image sequences and nonlinear ultrasound images of the nonlinear ultrasound image sequences into a new trajectory, further comprises:
calculating, by a processor a corresponding velocity for each microbubble trajectory based on tracking results;
if the absolute value of the velocity difference between two trajectories in the time-aligned linear ultrasound images of the linear ultrasound image sequence and nonlinear ultrasound images of the nonlinear ultrasound image sequence is less than a first preset threshold, and the average Euclidean distance of the point-by-point positions of the two trajectories is less than a second preset threshold, then determining the two trajectories as the duplicate microbubble trajectories and integrating them into a new trajectory.

26. The microvascular blood flow ultrasound imaging method of claim 24, wherein when sequentially performing nonlinear filtering processing on each group of echo signals in the echo signal group sequence, the method further comprising:
performing Fourier transform on each group of echo signals: P₁[ω]=Σₙ₌₀^{N}p₁[n]e^{-jωn}, P₂[ω]=Σₙ₌₀^{N}p₂[n]e^{-jωn}, where p₁[n] and p₂[n] are echo signals after two transmissions of ultrasound waves respectively, ω is a discrete frequency, n is a discrete time, and N is a number of sampling points for each reception;
extracting the fundamental frequency and its nearby components P′₁[ω] and P′₂[ω] of the Fourier transformed echo signals: P′₁[ω]=P₁[ω]|_{ωϵ[ω}_{₀}_{-Δω,ω}_{₀}_{+Δω]}, P′₂[ω]=P₂[ω]|_{ωϵ[ω}_{₀}_{-Δω,ω}_{₀}_{+Δω]}, where ω₀ is the fundamental frequency during transmission and reception, and Δω is the half bandwidth;
using the least squares method or gradient descent method to calculate the fundamental wave amplitude correction coefficient θ_{ω}_{₀} between the echo signals of the set that minimizes the difference Σ₁₀₇(P′₁[ω]−θ_{ω}_{₀}P′₂[ω])² between P′₁[ω] P′₂[ω], and applying the fundamental wave amplitude correction coefficient θ_{ω}_{₀}, to p₂[n] to obtain p′₂[n]=θ_{ω}_{₀}p₂[n], denoted as the echo signal after fundamental wave amplitude correction of p₂[n], where ωₛ is the maximum sampling frequency.

27. The microvascular blood flow ultrasound imaging method of claim 23, wherein the acquiring multiple groups of echo signals within a preset time period to obtain filtered and beamformed linear ultrasound image sequences and nonlinear ultrasound image sequences, further comprises:
imaging based on echoes, and acquiring an ultrasound image sequence within a preset time period;
using a linear filter to sequentially perform linear filtering processing on each frame of the ultrasound image sequence to obtain a corresponding linear ultrasound image sequence, and using a nonlinear filter to sequentially perform nonlinear filtering processing on each frame of the ultrasound image sequence to obtain a nonlinear ultrasound image sequence.

28. The microvascular blood flow ultrasound imaging method of claim 27, wherein the determining and integrating duplicate microbubble trajectories in the time-aligned linear ultrasound images of the linear ultrasound image sequences and nonlinear ultrasound images of the nonlinear ultrasound image sequences into a new trajectory, further comprises:
calculating a corresponding velocity for each microbubble trajectory based on tracking results; if the absolute value of the velocity difference between two trajectories in the time-aligned linear ultrasound images of the linear ultrasound image sequence and nonlinear ultrasound images of the nonlinear ultrasound image sequence is less than a first preset threshold, and the average Euclidean distance of the point-by-point positions of the two trajectories is less than a second preset threshold, then determining the two trajectories as the duplicate microbubble trajectories and integrating them into a new trajectory.

29. The microvascular blood flow ultrasound imaging method of claim 27, wherein the using a nonlinear filter to sequentially perform nonlinear filtering processing on each frame of the ultrasound image sequence to obtain a nonlinear ultrasound image sequence, further comprises:
performing Fourier transform in the axial direction on each frame of image obtained by beam synthesis: M₁[ω]=Σₙ₌₀^{N}m₁[n]e^{-jωn}, M₂[ω]=Σₙ₌₀ ^{N}m₂[n]e^{-jωn}, where m₁[n] and m₂[n] are the column signals of the images after two transmissions of ultrasound waves respectively, ω is the discrete frequency, n is the discrete time, and N is the number of sampling points for each reception;
extracting the fundamental frequency and its nearby components M′₁[ω] and M′₂[ω] of the Fourier transformed column (axial) of the image: M′₁[ω]=M′₁[ω]|_{ωϵ[ω}_{₀}_{-Δω,ω}_{₀}_{+Δω]}, M′₂[ω]=M′₂[ω]|_{ωϵ[ω}_{₀}_{-Δω,ω}_{₀}_{+Δω]}, where w₀ is the fundamental frequency during transmission and reception, and Aw is the half bandwidth;
using the least squares method or gradient descent method to calculate the fundamental wave amplitude correction coefficient θ_{ω}_{₀}, between the column (axial) of the set of images that minimizes the difference Σ_{ω}(M′₁[ω]−θ_{ω}_{₀} M′₂[ω])² between M′₁[ω] and M′₂[ω], and applying the fundamental wave amplitude correction coefficient θ_{ω}_{₀} to m₂[n] to obtain m′₂[n]=θ_{ω}_{₀}m₂[n], denoted as the column signal of the image after fundamental wave amplitude correction of m₂[n], where ωₛ is the maximum sampling frequency.

30. The microvascular blood flow ultrasound imaging method of claim 23, wherein the nonlinear imaging sequence comprises linear sequence and modulation sequence pairs, the modulation sequence in the linear sequence and modulation sequence pair is obtained by performing a preset modulation method on the linear sequence, wherein the preset modulation method comprises one or more of the following: pulse inversion, amplitude modulation, amplitude-phase modulation.

31. The microvascular blood flow ultrasound imaging method of claim 23, wherein the nonlinear imaging sequence comprises multiple identical pulse signals;
when transmitting the contrast pulse sequence to the imaging area, further comprising: dividing ultrasound array elements into multiple groups, and transmitting the multiple identical pulse signals to the imaging area by alternating transmission of the multiple groups.

32. The microvascular blood flow ultrasound imaging method of claim 23, wherein the sampling frequency for transmitting the contrast pulse sequence and receiving echoes comprises the Nyquist frequency.

33. A microvascular blood flow ultrasound imaging device, comprising:
a memory, for storing computer executable instructions; and,
a processor, for implementing the steps in the method of claim 23 when executing the computer executable instructions.

34. A non-transitory computer-readable storage medium, wherein the non-transitory computer-readable storage medium stores computer executable instructions, and when the computer executable instructions are executed by a processor, the steps in the method of claim 23 are implemented.
